(19) United States
(12) Patent Application Publication
(10) Pub. No.: US 2020/0323699 A1
(43) Pub. Date: Oct. 15, 2020

(54) Medication Delivery Device and Method

(71) Applicant: Herphoric, LLC, Fairfield, OH (US)

(72) Inventor: Paul BUSS, San Diego, CA (US)

(73) Assignee: Herphoric, LLC, Fairfield, OH (US)

(21) Appl. No.: 16/843,434

(22) Filed: Apr. 8, 2020

Related U.S. Application Data
(60) Provisional application No. 62/832,724, filed on Apr. 11, 2019.

(51) Int. Cl. A61F 13/20; A61K 9/48; A61K 9/00; A61K 31/05; A61K 31/352

(52) U.S. Cl. CPC A61F 13/2071; A61F 13/2074; A61K 9/4808; A61F 2013/15097; A61K 31/05; A61K 31/352; A61K 9/0034

(57) ABSTRACT

The disclosure relates to a device for vaginal drug delivery. More particularly, the device relates to a tampon engaged or engageable device, having a dissolvable elongated housing which is adapted with an axial cavity sized to surround a tampon, and having a fixed or removably engaged dissolvable capsule located at a distal end of the housing. So positioned, the device delivers medications such as prescription and non prescription drugs, neuroceuticals, and supplements and the like, directly to the vaginal tract which is especially useful in treating ailments in the same region by such a direct communication of the medication thereto.

### CROSS REFERENCE TO RELATED APPLICATIONS

The current application claims priority to U.S. provisional patent application Ser. No. 62/832,724, filed Apr. 11, 2019, the entire disclosure of which is incorporated herein.

### BACKGROUND

In modern medicine, a primary goal in the administration of medication is to deliver such medication to the body of the individual by the most direct route to the target of the medication. In other words, delivery of a drug to treat a medical ailment of a patient should be highly targeted to reach and treat the ailment for which the medication is intended in the least amount of time, with the least amount of communication of the medication administered to areas of the body not requiring it. Such accurate targeting is especially desired where other parts of the body may react adversely to the medication, or where a circuitous route through the body may dilute the medication.

In the case of vaginal disorders which may occur monthly such as cramps or which may be caused by viruses, yeast, or other pathogens, it is desirable that a treating medication be delivered directly to the vaginal cavity of the person. Further, in some cases, it is desirable that the medication delivery be prolonged so as to have time to communicate to regions of the vaginal canal or cervix without problems from body fluids draining from the delivery area preventing medication delivery.

Multiple delivery systems have been employed over recent decades to provide such targeted and prolonged delivery. However, because of issues with drug delivery via the vaginal canal, suppositories and foams which provide carriers for the medication frequently fail or are rendered less than effective due to fluid flow and drainage from the canal. Tampon related devices have been tried, but the prior art is configured in such a manner that the released medication can actually be absorbed by the tampon rather than delivered to the body of the user.

The forgoing prior art examples concerning vaginal drug delivery are intended to be illustrative and not exclusive, and they do not imply any limitations on the invention described and claimed herein. Various other limitations of the related art are known or will become apparent to those skilled in the art upon a reading and understanding of the specification below and the accompanying drawings.

### SUMMARY

The device and method herein provide a solution to the targeted delivery of medications to targeted areas within or in direct communication with the vagina. Further, the system herein is employable for targeted delivery of medication, over a prolonged time period, through direct communication through the tissue of the vaginal canal to treat ailments directly, and to also communicate medications and the like to the bloodstream and surrounding areas.

The disclosed system relates to a device for vaginal drug delivery. More particularly, the system relates to a tampon engaged or engageable device, having a dissolvable elongated housing which is adapted with an axial cavity sized to surround a tampon, and having a fixed or removably engaged dissolvable capsule located at a distal end of the housing. So positioned, the device delivers medications such as prescription and non prescription drugs, neuroceuticals, and supplements and the like, directly to the vaginal tract which is especially useful in treating ailments in the same region by such a direct communication of the medication thereto.

The exemplary device herein provides an easy to employ vaginal drug delivery system, through the employment of a medication infused polymeric capsule and/or cylindrical housing which is adapted for employment in combination with a tampon, or provided in an assembled form therewith. The product is thus employable to communicate medications directly to the patient through the vaginal canal, where it can directly communicate and treat a related ailment, prior to the tampon becoming active. So configured, the device can be employed for delivery of analgesics, antifungal medications, newer medications such as CBD and THC, and any other medication where communication thereof in a targeted fashion through the vaginal canal is desired.

Because tampons are widely employed throughout the world, the provision of the exemplary device in configurations adapted for engagement to a tampon, or in a combination with a tampon, help insure widespread use. This is because widespread familiarity with the use and employment of tampons does not require most users to change their normal habits or learn new skills. Further, the device and method herein are configured to disburse the medication to the vaginal tract before the tampon is placed in active and absorbing communication therewith.

In one mode, the device may be provided as a capsule adapted for engagement to, or provided in combination with, a cylindrical housing having an axial cavity housing a tampon. The capsule may be a dissolvable polymer such as PCL or other material, which is impregnated with, and/or has a cavity therein, which holds the medications to be delivered to the targeted area via the vaginal canal. The cylindrical housing may also be formed of such dissolvable material which is preferably configured to dissolve more slowly than the material forming the capsule. This intentional delay for a calculated duration, allows dissolving of the capsule with concurrent communication of the medication therein to the body, before the cylindrical housing dissolves and exposes the absorbent tampon. It thus prevents absorption of the medication by the tampon.

In a similar mode of the device herein, the capsule and cylindrical housing can be formed as a unitary structure. In this mode the capsule may dissolve at a concurrent rate as the dissolution of the housing, or it could dissolve more rapidly thereby positioning the cylindrical housing surrounding the tampon to inhibit absorption of the medication.

In another mode of the device herein, the capsule may be configured in the same dissolvable manner so as to provide ongoing delivery of medication locally. However, the dissolvable capsule may be adapted for a removable engagement to a conventional plastic or paper housing which surrounds a conventional tampon. In use, the capsule, in this configuration, will disengage and dissolve over time. Such will allow for the capsules bearing the medication required for the patient to be dispensed from a pharmacy, and engaged to conventional tampons whereupon they would release on insertion and dissolve over time.

With respect to the above description, before explaining at least one preferred embodiment of the herein disclosed vaginal drug delivery system invention in detail, it is to be understood that the invention is not limited in its application to the details of construction and to the arrangement of the components in the following description or illustrated in the drawings. The invention herein described and shown is capable of other embodiments and of being practiced and carried out in various other ways by those skilled in the art upon reading this disclosure. Also, it is to be understood that the phraseology and terminology employed herein are for the purpose of description and should not be regarded as limiting.

As such, those skilled in the art will appreciate that the conception upon which this disclosure is based may readily be utilized as a basis for designing of other vaginal drug delivery devices and for carrying out the several purposes of the present disclosed device. It is important, therefore, that the claims be regarded as including such equivalent construction and methodology insofar as they do not depart from the spirit and scope of the present invention.

As used in the claims to describe the various inventive aspects and embodiments, “comprising” means including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements. The term “substantially” when employed herein, means plus or minus twenty percent unless otherwise designated in range.

It is an object of the present disclosure to provide an easily employed vaginal drug delivery system and method therefor.

It is an additional object of this disclosure to provide a dissolvable drug delivery device which is adapted for engagement around a tampon or provided in combination with a tampon, to provide for easy and widespread use.

These and other objects, features, and advantages of the present vaginal drug delivery device and method in the system herein, as well as the advantages thereof over existing prior art, which will become apparent from the description to follow, are accomplished by the improvements described in this specification and hereinafter described in the following detailed description which fully discloses the invention, but should not be considered as placing limitations thereon in any fashion.

### BRIEF DESCRIPTION OF DRAWING FIGURES

The accompanying drawings, which are incorporated herein and form a part of the specification, illustrate some, but not the only or exclusive examples of embodiments and/or features of the disclosed system employing the device and method herein. It is intended that the embodiments and figures disclosed herein are to be considered illustrative of the invention herein, rather than limiting in any fashion.

In the drawings:

FIG. 1 depicts an exterior view of one mode of the device herein which formed in a unitary structure which is adapted for engagement with a tampon or which may be provided in combination therewith.

FIG. 2 is a sectional view through the device of FIG. 1.

FIG. 3 shows another mode of the device herein which employs a drug delivery capsule which is removably engageable to a dissolvable cylindrical housing or adapted for engagement to a conventional tampon cartridge, thereby allowing for pharmacy sale of multiple types of capsules with one or a plurality of medications therein, which may be engaged to the housing.

FIG. 4 is a sectional view through the device of FIG. 3.

FIG. 5 depicts a mode of the device where the dissolving capsule is configured with a slot or opening adapted to engage with a pill or capsule type medication.

FIG. 6 depicts the capsule or pill type medication in a removable engagement within a slot or opening in the dissolving capsule.

### DETAILED DESCRIPTION

In this description, the directional prepositions of up, upwardly, down, downwardly, front, back, top, upper, bottom, lower, left, right and other such terms refer to the device as it is oriented and appears in the drawings and are used for convenience only and such are not intended to be limiting or to imply that the device has to be used or positioned in any particular orientation. Additionally, where the word substantially is used, such is intended to be plus or minus twenty percent, unless otherwise defined upon such use.

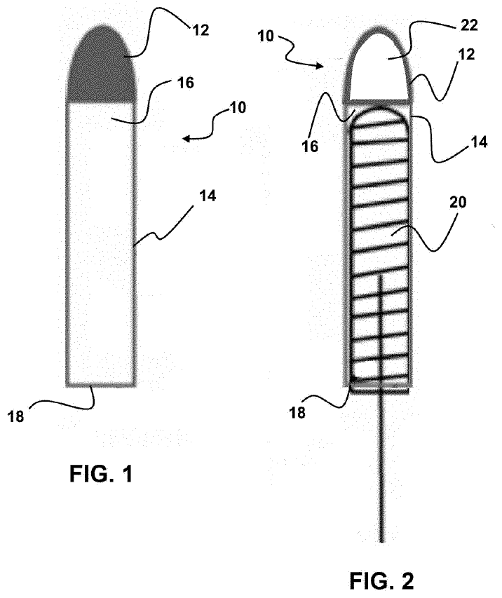

Now referring to drawings in FIGS. 1-6, wherein similar components are identified by like reference numerals, there is seen in FIG. 1 a mode the device 10 formed in a unitary structure. In the mode of FIGS. 1-2, a capsule 12 is engaged to the distal end of a cylindrical housing 14.

An axial cavity 16 is positioned within the sidewall forming the cylindrical housing 14 and has an opening 18 communicating with the axial cavity 16 at a proximal end. The opening 18 allows for the insertion of a tampon 20 into the axial cavity 16.

In the mode of the device 10 of FIGS. 1-2, the capsule 12 is formed of a fluid-dissolvable material such as PCL, as is the sidewall forming the cylindrical housing 14. The dissolvable material forming one or both of the capsule 12 and cylindrical housing 14 may be impregnated with the desired medication or medications to be delivered to the vaginal canal during the dissolving of the capsule 12 and the cylindrical housing 14.

In a preferred mode to help prevent absorption of the fluid bearing the medication delivered by the dissolving capsule 12, the wall forming the cylindrical housing 14 is formed of a material mix calculated to dissolve more slowly than the capsule 12. In this fashion the medication from the capsule 12 is communicated to the vaginal canal before the tampon 20 is exposed to fluid flow therein. As noted, this prevents absorption of the medication by the tampons.

The capsule 12 may be formed in a solid solution of dissolvable polymer or other material which is impregnated with the medication, or it may be formed with a capsule cavity 22 which is surrounded by walls forming the capsule 12. Where the capsule cavity 22 is provided, dissolvable medication may be positioned within the capsule cavity 22 which will disperse into the vaginal canal once an opening forms in the wall of the capsule 12 as it dissolves.

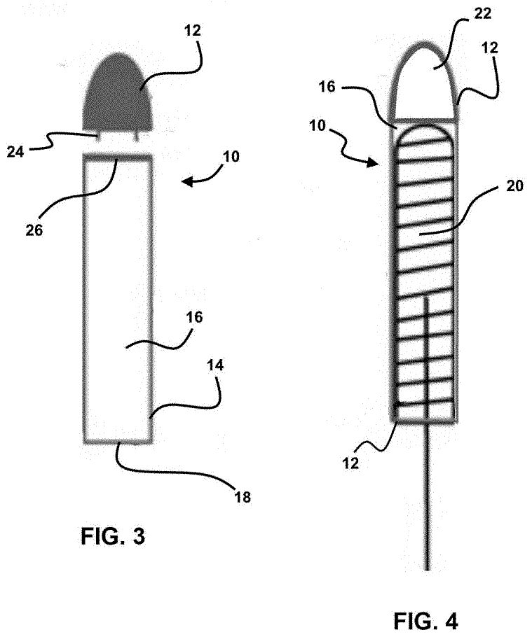

As depicted in FIGS. 3-4, the device 10 may be formed with an engageable capsule 12, which is adapted to engage with a distal end of a cylindrical housing 14. This mode allows for doctors and pharmacies to stock multiple medications which are preferably delivered through the vaginal cavity, and engage them or provide them to the patient for engagement to the distal end of a cylindrical housing 16 surrounding a tampon 20. As can be discerned, capsules 12 having an infinite number of different medications may be stocked and provided which treat specific patient ailments, and may be engaged to a stock cylindrical housing 14. This allows for high customability of the capsules 12 which are impregnated with one or a plurality of medications which dissolve as the capsule dissolves.

Additionally, capsules 12 having a secondary supply of one or a plurality of medications may be provided where the capsule 12 is formed with a capsule cavity 22. This secondary medication supply may be pre-positioned within the capsule cavity 22, or for example, may be positioned therein by a medical professional using, for example, a hypodermic needle which is temporarily communicated through the wall of the capsule 12 surrounding the capsule cavity 22.

Finally, the removably engageable dissolving capsule 12 may be configured with mating connectors 24 or adhesive, which is configured to engage on or with the distal end of the cylindrical housing 14. Such, for example, might be pins or hooks which engage with the internally housed tampon 20, or might be projections which engage with connectors positioned on an endwall 26 at the distal end of the cylindrical housing 14. Where adhesive provides the mating connectors 24, such may engage the endwall 26 or might engage the internal surface of the axial cavity 16 if no endwall 26 is present.

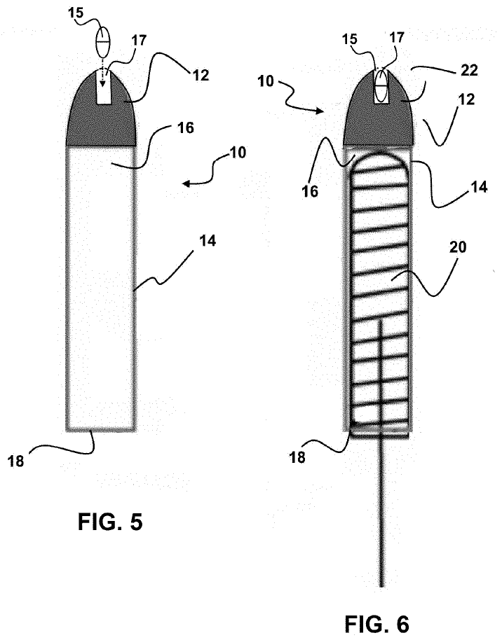

Finally, FIGS. 5-6 depict a mode of the device 10 herein where the dissolving capsule 12 is adapted for an engagement with a medication such as the depicted tablet or capsule 15. In this mode, the tablet or capsule 15 of medication or supplement of choice shown in FIG. 5, is positioned within a slot 17 in the dissolvable capsule 12 which is adapted to engage it. Such might be, for example, by forming the sidewalls of the slot 17 to be flexible and forming the width of the slot smaller than most conventionally available tablets and capsules 15 used for medications and supplements. In use, the tablet or capsule 15 is slid into a compressive engagement within the slot 17 such as shown in FIG. 6. The medication and/or supplement provided in the capsule 15 will thus be delivered to the vaginal canal where it can dissolve.

It should be noted that any of the different depicted and described configurations and components of the device 10 employed in the system herein for vaginal drug delivery, can be employed with any other configuration or component shown and described as part of the device herein. Additionally, while the present invention has been described herein with reference to particular embodiments thereof and/or steps in the method of production or use, a latitude of modifications, various changes and substitutions are intended in the foregoing disclosure, and it will be appreciated that in some instance some features, or configurations, of the invention could be employed without a corresponding use of other features without departing from the scope of the invention as set forth in the following claims. All such changes, alternations and modifications as would occur to those skilled in the art are considered to be within the scope of this invention as broadly defined in the appended claims.

What is claimed is:

1. A medication delivery device comprising:
a dissolvable housing formed from a first dissolvable material that is dissolvable in the presence of fluid, having an elongated cylindrical wall with an axial cavity extending therethrough, a trailing end portion, and a leading end portion, the axial cavity is dimensionally sized to receive a tampon;
a dissolvable capsule formed from a second dissolvable material that is dissolvable in the presence of fluid, having a trailing end portion that is coupled to the leading end portion of the dissolvable housing, the dissolvable capsule comprising at least one wall enclosing a capsule cavity therein;
wherein at least one of the first dissolvable material and the second dissolvable material is impregnated with at least one medication.

2. The medication delivery device of claim 1, wherein the first dissolvable material and the second dissolvable material are configured to dissolve at a concurrent rate.

3. The medication delivery device of claim 1, wherein the first dissolvable material is configured to dissolve more slowly than the second dissolvable material.

4. The medication delivery device of claim 1, wherein at least one of the first dissolvable material and the second dissolvable material comprises a dissolvable polymer.

5. The medication delivery device of claim 1, wherein the capsule cavity contains a medication.

6. The medication delivery device of claim 1, wherein the first dissolvable material comprises a first medication impregnated therein and the second dissolvable material comprises a second medication impregnated therein, different than the first medication.

7. The medication delivery device of claim 1, further comprising a slot formed in a periphery of the lead portion of the dissolvable capsule configured to removably engage a medication delivery mechanism.

8. The medication delivery device of claim 7, wherein the medication delivery mechanism is at least one of a medicine tablet or a medicine capsule.

9. The medication delivery device of claim 1, wherein the dissolvable capsule is removably coupled to the dissolvable housing.

10. The medication delivery device of claim 9, comprising a plurality of dissolvable capsules, each containing a different medication, that may each be selectively coupled to the dissolvable housing depending upon a desired treatment.

11. The medication of claim 1, wherein the impregnated medication comprises at least one of an antifungal medication, a CBD medication or a THC medication.

12. A medication device comprising:
a dissolvable housing formed from a first dissolvable material that is dissolvable in the presence of fluid, having an elongated cylindrical wall with an axial cavity extending therethrough, a trailing end portion, and a leading end portion, the axial cavity is dimensionally sized to receive a tampon;
a dissolvable capsule formed from a second dissolvable material that is dissolvable in the presence of fluid, having a trailing end portion that is coupled to the leading end portion of the dissolvable housing, the dissolvable capsule comprising at least one of a capsule cavity or a capsule slot for receiving at least one first medication.

13. The medication delivery device of claim 12, wherein the first dissolvable material and the second dissolvable material are configured to dissolve at a concurrent rate.

14. The medication delivery device of claim 12, wherein the first dissolvable material is configured to dissolve more slowly than the second dissolvable material.

15. The medication delivery device of claim 12, wherein at least one of the first dissolvable material and the second dissolvable material comprises a dissolvable polymer.

16. The medication delivery device of claim 12, wherein at least one of the first dissolvable material and the second dissolvable material comprises at least a second medication impregnated therein.

17. The medication delivery device of claim 16, wherein the first dissolvable material comprises the second medication impregnated therein and the second dissolvable material comprises a third medication impregnated therein, different than the second medication.

18. The medication delivery device of claim 12, wherein the first medication is in the form of a tablet or a medicine capsule received within a capsule slot in the dissolvable capsule.

19. The medication delivery device of claim 12, wherein the dissolvable capsule is removably coupled to the dissolvable housing.

20. The medication delivery device of claim 19, comprising a plurality of dissolvable capsules, each containing a different medication in the capsule cavity or capsule slot, that may each be selectively coupled to the dissolvable housing depending upon a desired treatment.

21. The medication delivery device of claim 19, wherein the dissolvable capsule is removably coupled to the dissolvable housing using at least one of an adhesive, a projection or a mating connector.

22. The medication of claim 12, wherein the first medication comprises at least one of an antifungal medication, a CBD medication or a THC medication.

23. A medication delivery device comprising:
a dissolvable housing formed from a first dissolvable material that is dissolvable in the presence of fluid, having an elongated cylindrical wall with an axial cavity extending therethrough, a trailing end portion, and a leading end portion, the axial cavity is dimensionally sized to receive a tampon;
wherein the first dissolvable material is impregnated with at least one medication comprising at least one of an antifungal medication, a CBD medication or a THC medication.

24. The medication delivery device of claim 22, wherein the first dissolvable material is configured to dissolve at a first predetermined rate.

25. The medication delivery device of claim 22, wherein the first dissolvable material further comprises a dissolvable polymer.

26. A method for delivery of a medication to a patient comprising:
providing medication delivery device including a dissolvable housing formed from a first dissolvable material that is dissolvable in the presence of fluid, having an elongated cylindrical wall with an axial cavity extending therethrough, a trailing end portion, and a leading end portion, the axial cavity is dimensionally sized to receive a tampon, wherein the first dissolvable material is impregnated with at least one medication comprising at least one of an antifungal medication, a CBD medication or a THC medication;
inserting a tampon in the axial cavity; and
placing the medication delivery device in the patient's vaginal canal so that vaginal fluids will dissolve at least a portion of the dissolvable material allowing the medication to be delivered to the patient through the vaginal canal as the dissolvable materials dissolves therein.

27. A method for delivery of a medication to a patient comprising:
providing a medication delivery device including (a) a dissolvable housing formed from a first dissolvable material that is dissolvable in the presence of fluid, having an elongated cylindrical wall with an axial cavity extending therethrough, a trailing end portion, and a leading end portion, the axial cavity is dimensionally sized to receive a tampon, (b) a dissolvable capsule formed from a second dissolvable material that is dissolvable in the presence of fluid, having a trailing end portion that is coupled to the leading end portion of the dissolvable housing, the dissolvable capsule comprising at least one wall enclosing a capsule cavity therein, wherein at least one of the first dissolvable material and the second dissolvable material is impregnated with at least one first medication;
inserting a tampon in the axial cavity; and
placing the medication delivery device in the patient's vaginal canal so that vaginal fluids will dissolve at least a portion of the first and second dissolvable materials allowing the at least one first medication to be delivered to the patient through the vaginal canal as the first and second dissolvable materials dissolves therein.

28. The method of claim 27, wherein the first dissolvable material and the second dissolvable material are configured to dissolve at a concurrent rate.

29. The method of claim 27, wherein the first dissolvable material is configured to dissolve more slowly than the second dissolvable material.

30. The method of claim 27, wherein at least one of the first dissolvable material and the second dissolvable material comprises a dissolvable polymer.

31. The method of claim 27, wherein the capsule cavity contains a second medication, wherein dissolving of the second dissolvable material allows the second medication to be delivered to the patient through the vaginal canal as the second dissolvable materials dissolves therein.

32. The method of claim 27, wherein the first dissolvable material comprises the first medication impregnated therein and the second dissolvable material comprises a second medication impregnated therein, different than the first medication, so that vaginal fluids will dissolve at least a portion of the first and second dissolvable materials allowing the first and second medications to be delivered to the patient through the vaginal canal as the first and second dissolvable materials dissolves therein.

33. The method of claim 27, further comprising a slot formed in a periphery of the lead portion of the dissolvable capsule configured to removably engage a medication delivery mechanism.

34. The method of claim 33, wherein the medication delivery mechanism is at least one of a medicine tablet or a medicine capsule.

35. The method of claim 27, wherein the dissolvable capsule is removably coupled to the dissolvable housing and the method includes selectively coupling the dissolvable capsule to the dissolvable housing before the placing step.

36. The method of claim 35, comprising a plurality of dissolvable capsules, each containing a different medication, that may each be selectively coupled to the dissolvable housing depending upon a desired treatment.
